SEQUENCE LISTING (9 sequences)

SEQ ID NO: 1 - protein, 89 aa, Homo sapiens
  MISC_FEATURE (1)..(89): HUMAN CXCL12/SDF-1 Alpha
  ref <308>: DATABASE ACCESSION NUMBER: NCBI Protein/NP_954637.1
  ref <309>: DATABASE ENTRY DATE: 2021-03-29
  ref <313>: RELEVANT RESIDUES IN SEQ ID NO: (1)..(89)
MNAKVVVVLVLVLTALCLSDGKPVSLSYRCPCRFFESHVARANVKHLKILNTPNCALQIVARLKNNNRQVCIDPKLKWIQEYLEKALNK

SEQ ID NO: 2 - protein, 93 aa, Homo sapiens
  MISC_FEATURE (1)..(93): HUMAN CXCL12/SDF-1 Beta
  ref <308>: DATABASE ACCESSION NUMBER: UniPrtoKB/P48061.1
  ref <309>: DATABASE ENTRY DATE: 2021-02-10
  ref <313>: RELEVANT RESIDUES IN SEQ ID NO: (1)..(93)
MNAKVVVVLVLVLTALCLSDGKPVSLSYRCPCRFFESHVARANVKHLKILNTPNCALQIVARLKNNNRQVCIDPKLKWIQEYLEKALNKRFKM

SEQ ID NO: 3 - protein, 119 aa, Homo sapiens
  MISC_FEATURE (1)..(119): HUMAN CXCL12/SDF-1 Gamma
  ref <308>: DATABASE ACCESSION NUMBER: NCBI Protein/NP_001029058.1
  ref <309>: DATABASE ENTRY DATE: 2021-03-29
  ref <313>: RELEVANT RESIDUES IN SEQ ID NO: (1)..(119)
MNAKVVVVLVLVLTALCLSDGKPVSLSYRCPCRFFESHVARANVKHLKILNTPNCALQIVARLKNNNRQVCIDPKLKWIQEYLEKALNKGRREEKVGKKEKIGKKKRQKKRKAAQKRKN

SEQ ID NO: 4 - protein, 140 aa, Homo sapiens
  MISC_FEATURE (1)..(140): HUMAN CXCL12/SDF-1 Delta
  ref <301>: AUTHORS: Yu, L. et al.
  ref <302>: TITLE: Identification and expression of novel isoforms of human stromal cell-derived factor 1
  ref <303>: JOURNAL: Genes and Development
  ref <304>: VOLUME: 374
  ref <306>: PAGES: 174-9
  ref <307>: DATE: 2006-06-07
  ref <313>: RELEVANT RESIDUES IN SEQ ID NO: (1)..(140)
MNAKVVVVLVLVLTALCLSDGKPVSLSYRCPCRFFESHVARANVKHLKILNTPNCALQIVARLKNNNRQVCIDPKLKWIQEYLEKALNNLISAAPAGKRVIAGARALHPSPPRACPTARALCEIRLWPPPEWSWPSPGDV

SEQ ID NO: 5 - protein, 90 aa, Homo sapiens
  MISC_FEATURE (1)..(90)
  ref <301>: AUTHORS: Yu, L. et al.
  ref <302>: TITLE: Identification and expression of novel isoforms of human stromal cell-derived factor 1
  ref <303>: JOURNAL: Genes and Development
  ref <304>: VOLUME: 374
  ref <306>: PAGES: 174-9
  ref <307>: DATE: 2006-06-07
  ref <313>: RELEVANT RESIDUES IN SEQ ID NO: (1)..(90)
MNAKVVVVLVLVLTALCLSDGKPVSLSYRCPCRFFESHVARANVKHLKILNTPNCALQIVARLKNNNRQVCIDPKLKWIQEYLEKALNNC

SEQ ID NO: 6 - protein, 100 aa, Homo sapiens
  MISC_FEATURE (1)..(100): HUMAN CXCL12/SDF-1 Phi
  ref <301>: AUTHORS: Yu, L. et al.
  ref <302>: TITLE: Identification and expression of novel isoforms of human stromal cell-derived factor 1
  ref <303>: JOURNAL: Genes and Development
  ref <304>: VOLUME: 374
  ref <306>: PAGES: 174-9
  ref <307>: DATE: 2006-06-07
  ref <313>: RELEVANT RESIDUES IN SEQ ID NO: (1)..(100)
MNAKVVVVLVLVLTALCLSDGKPVSLSYRCPCRFFESHVARANVKHLKILNTPNCALQIVARLKNNNRQVCIDPKLKWIQEYLEKALNKIWLYGNAETSR

SEQ ID NO: 7 - DNA, 375 bp, Artificial Sequence
  CXCL12/SDF-1 beta + FLAG tag
  misc_feature (1)..(375): CXCL12/SDF-1 beta + FLAG tag
atgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagaggttcaagatgacgcgtacgcggccgctcgagcagaaactcatctcagaagaggatctggcagcaaatgatatcctggattacaaggatgacgacgataaggtttaa

SEQ ID NO: 8 - DNA, 363 bp, Artificial Sequence
  CXCL12/SDF-1 alpha + FLAG tag
  misc_feature (1)..(363): CXCL12/SDF-1 alpha + FLAG tag
atgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagacgcgtacgcggccgctcgagcagaaactcatctcagaagaggatctggcagcaaatgatatcctggattacaaggatgacgacgataaggtttaa

SEQ ID NO: 9 - protein, 8 aa, Artificial Sequence
  FLAG tag
  MISC_FEATURE (1)..(8): FLAG tag
DYKDDDDK

(19) United States
(12) Patent Application Publication
(10) Pub. No.: US 2023/0149511 A1
(43) Pub. Date: May 18, 2023

(54) TREATING AUTOIMMUNE DISEASES WITH GENETICALLY MODIFIED CELLS

(71) Applicant: WALLKILL BIOPHARMA, INC., Rancho Santa Fe, CA (US)

(72) Inventors: Lisa HULLE, Rancho Santa Fe, CA (US); David KIEWLICH, Rancho Santa Fe, CA (US); Joan ROBBINS, Rancho Santa Fe, CA (US); Gerald SWISS, Rancho Santa Fe, CA (US)

(21) Appl. No.: 17/995,847

(22) PCT Filed: Apr. 9, 2021
(86) PCT No.: PCT/US2021/026678

§ 371 Date: Oct. 7, 2022

Related U.S. Application Data
(60) Provisional application No. 63/007,796, filed on Apr. 9, 2020.

(51) Int. Cl. A61K 38/19; A61K 48/00; A61K 35/17; A61P 37/00

(52) U.S. Cl. CPC A61K 38/195; A61K 48/0025; A61K 35/17; A61P 37/00

(57) ABSTRACT

Described herein are human genetically modified cells or precursors expressing fugetactic levels of a fugetactic agent, e.g. CXCL12, and methods of treating an autoimmune disease in a subject in need thereof. Also described herein are cells or precursors comprising a transgene or other genetic modification for expression of a nucleic acid sequence encoding a fugetactic agent, e.g. CXCL12.

### SEQUENCE LISTING

This application claims priority to U.S. Provisional Application No. 63/007,796, filed Apr. 9, 2020, which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Apr. 9, 2021, is named 054610-506001W0 ST25.txt and is 9,219 bytes in size.

### FIELD OF THE INVENTION

The invention is directed to genetically modified cells (e.g., human thyroid cells, hepatocytes, endothelial cells, epithelial cells, joint cells, cortisol/aldosterone-producing cells, or precursors or stem cells) as well as methods using such cells, e.g. for treatment of autoimmune diseases. The genetically modified (transgenic) cells express a fugetactic amount of a fugetactic agent thereby imparting protection against human mononuclear immune cells. In one embodiment, the fugetactic agent is, for example, CXCL12. In one embodiment, the genetically modified cells comprise a vector, wherein the vector comprises a nucleic acid sequence encoding a fugetactic agent and preferably a human fugetactic agent. In one embodiment, the genetically modified cells are further modified to be senescent. Methods of this invention include use of these cells in autoimmune disease patients.

### BACKGROUND OF THE INVENTION

Thyroid cells are responsible for the production of thyroid hormones thyroxine and triiodothyronine, proteins believed to play an important role in thyroid function. Cortisol/aldosterone-producing cells are responsible for the production of cortisol and aldosterone, proteins believe to play an important role in adrenal function. Hepatocytes are responsible for the production of bile, and are believe to play an important role in liver function. Endothelial cells are responsible for many aspects of vascular biology and are believed to play an important role in controlling the passage of materials into and out of the bloodstream. Epithelial cells are responsible for lining the surface of body cavities and hollow organs, and are believed to play an important role in forming glands and protecting organs.

An autoimmune disease develops when the body's immune system fails to recognize normal body tissues and attacks and destroys them as if they were foreign. There are many autoimmune diseases with symptoms that range from mild rashes to life-threatening conditions that attack major organ systems. Though each disease is different, immune-system malfunction is present in all of them. Disease symptoms vary depending on which tissue is targeted for destruction. Autoimmune disorders are frequently classified into organ-specific, or localized, disorders and non-organ-specific types. In organ-specific disorders, the autoimmune process is directed mostly against one organ. But patients may experience several organ-specific diseases at the same time. The causes of autoimmune disorders are not well understood and many have no cure.

In view of the above, there is a long unmet need to develop technology that effectively treats autoimmune diseases.

### SUMMARY OF THE INVENTION

This invention is directed to genetically modified cells (e.g., human thyroid cells or precursors as well as genetically modified, senescent, human thyroid cells or precursors, or stem cells) that express an effective amount of a fugetactic agent so as to render these cells resistant to human immune cells. This disclosure is also directed to methods of using the cells, for example for treating autoimmune diseases, e.g. autoimmune disease that attacks an organ or tissue.

This invention is also directed to genetically modified, human cortisol/aldosterone-producing cells or precursors as well as genetically modified, senescent, human cortisol/aldosterone-producing cells or precursors that express an effective amount of a fugetactic agent so as to render these cells resistant to human immune cells. This invention is also directed to genetically modified, human hepatocyte cells or precursors as well as genetically modified, senescent, human hepatocyte cells or precursors that express an effective amount of a fugetactic agent so as to render these cells resistant to human immune cells. This invention is also directed to genetically modified, human endothelial cells or precursors as well as genetically modified, senescent, human endothelial cells or precursors that express an effective amount of a fugetactic agent so as to render these cells resistant to human immune cells. This invention is also directed to genetically modified, human epithelial cells or precursors as well as genetically modified, senescent, human epithelial cells or precursors that express an effective amount of a fugetactic agent so as to render these cells resistant to human immune cells. This invention is also directed to genetically modified, human stem cells that express an effective amount of a fugetactic agent so as to render these cells resistant to human immune cells. In embodiments, the stem cells are pluripotent or multipotent stem cells that are capable into differentiation into one or more cell types. In embodiments, the stem cells are capable into differentiation into a cell type that is targeted by immune cells (e.g., T cells) in an autoimmune disease.

Fugetactic agents are well known in the art, including CXCL12. This cytokine, also known as SDF-1, is produced by thymic and bone marrow stroma (see e.g. U.S. Pat. No. 5,756,084, entitled: “Human stromal derived factor 1α. and 1β.,” issued May 26, 1998, to Honjo, et al.). CXCL12 has been reported to repel effector T-cells while recruiting immune-suppressive regulatory T-cells to an anatomic site. See, e.g., Poznansky et al., Nature Medicine 2000, 6:543-8. CXCL12 and its receptor CXCR4 are also reported to be an integral part of angiogenesis.

Agents other than CXCL12 are also disclosed to repel immune cells, including, without limitation, other CXCR4 ligands, CXCR4-binding antibodies, and the like. Non-limiting examples of fugetactic (chemorepellant) proteins can be found in U.S. Pat. Nos. 7,745,578 and 9,617,330, each of which is incorporated herein by reference in its entirety.

An embodiment of the invention is a genetically modified human thyroid cell or precursor expressing an effective amount of a fugetactic agent, preferably CXCL12, so as to render the cell resistant to human immune cells. In one embodiment, such fugetactic effective amounts of the fugetactic agent are generated by introduction of a human transgene for the agent (e.g., CXCL12) into the thyroid cell or a precursor of the thyroid cell (e.g., a pluripotent stem cell, multipotent stem cell, etc.). These human genetically modified thyroid cells (e.g., follicular cells or parafollicular cells) or precursors are further characterized as expressing thyroid hormones thyroxine and triiodothyronine in response to pituitary hormones, or express thyroid hormones thyroxine and triiodothyronine after differentiation into follicular cells. An embodiment of the invention is a genetically modified human cortisol/aldosterone-producing cell or precursor expressing an effective amount of a fugetactic agent, preferably CXCL12, so as to render the cell resistant to human immune cells. In one embodiment, such fugetactic effective amounts of the fugetactic agent are generated by introduction of a human transgene for the agent (e.g., CXCL12) into the cortisol/aldosterone-producing cell or a precursor of the cortisol/aldosterone-producing cell (e.g., a pluripotent stem cell, multipotent stem cell, etc.). These human genetically modified cortisol/aldosterone-producing cells or precursors are further characterized as expressing cortisol or aldosterone in response to pituitary hormones, or express cortisol or aldosterone after differentiation into cortisol/aldosterone-producing cells. An embodiment of the invention is a genetically modified human hepatocyte cell or precursor expressing an effective amount of a fugetactic agent, preferably CXCL12, so as to render the cell resistant to human immune cells. In one embodiment, such fugetactic effective amounts of the fugetactic agent are generated by introduction of a human transgene for the agent (e.g., CXCL12) into the hepatocyte cell or a precursor of the hepatocyte cell (e.g., a pluripotent stem cell, multipotent stem cell, etc.). These human genetically modified hepatocyte cells or precursors are further characterized as expressing cholesterol, bile salts and/or phospholipids, or express cholesterol, bile salts and/or phospholipids after differentiation into hepatocyte cells. An embodiment of the invention is a genetically modified human endothelial cell or precursor expressing an effective amount of a fugetactic agent, preferably CXCL12, so as to render the cell resistant to human immune cells. In one embodiment, such fugetactic effective amounts of the fugetactic agent are generated by introduction of a human transgene for the agent (e.g., CXCL12) into the endothelial cell or a precursor of the endothelial cell (e.g., a pluripotent stem cell, multipotent stem cell, etc.). An embodiment of the invention is a genetically modified human epithelial cell or precursor expressing an effective amount of a fugetactic agent, preferably CXCL12, so as to render the cell resistant to human immune cells. In one embodiment, such fugetactic effective amounts of the fugetactic agent are generated by introduction of a human transgene for the agent (e.g., CXCL12) into the epithelial cell or a precursor of the epithelial cell (e.g., a pluripotent stem cell, multipotent stem cell, etc.). As such, these cells can be used in a method for treating an autoimmune disorder in a patient. In embodiments, the cells can be used for treating an autoimmune disorder that primarily affects a certain organ or tissue, e.g. Graves' Disease, Crohn's Disease, Addison's Disease, autoimmune hepatitis, Hashimoto's Thyroiditis, Reactive Arthritis, Giant-cell Arteritis (GCA), or celiac disease, in a subject. In an embodiment, the cells are not used to treat type 1 diabetes or multiple sclerosis in a subject.

The genetically modified cells used in the methods described herein may be autologous or non-autologous, e.g., allogenic.

In one embodiment, the patient suffers from Graves' Disease. In one embodiment, the patient suffers from Crohn's Disease. In one embodiment, the patient suffers from Addison's Disease, also known as primary adrenal insufficiency or autoimmune attack of the adrenal glands. In one embodiment, the patient suffers from autoimmune hepatitis, also known as autoimmune attack on the liver. In one embodiment, the patient suffers from Hashimoto's Thyroiditis, also known as chronic lymphotic thyroiditis or autoimmune attack of the thyroid. In one embodiment, the patient suffers from Reactive Arthritis, formerly known as Reiter's syndrome. In one embodiment, the patient suffers from Giant-cell Arteritis (GCA), also called temporal arthritis. In one embodiment, the patient suffers from celiac disease. In an embodiment, the patient does not have type 1 diabetes or multiple sclerosis.

In another embodiment, the genetically modified human cells can be modified to be senescent (incapable of division) such that any further differentiation of these cells into cancer cells is eliminated and apoptotic induction arising due to inappropriate cell division is negated.

An embodiment of this invention uses thyroid cells or precursors to increase levels of thyroid hormones thyroxine and triiodothyronine and reduce autoimmune response to the genetically modified cells.

An embodiment of this invention uses cortisol/aldosterone-producing cells or precursors to increase levels of cortisol or aldosterone and reduce autoimmune response to the genetically modified cells.

An embodiment of this invention uses hepatocyte cells or precursors to increase levels of bile and reduce autoimmune response to the genetically modified cells.

An embodiment of this invention uses endothelial cells or precursors to reduce autoimmune response to the genetically modified cells.

An embodiment of this invention uses epithelial cells or precursors to reduce autoimmune response to the genetically modified cells.

In an embodiment, the genetically modified cells are not oligodendrocytes (or oligodendrocyte precursors. In an embodiment, the genetically modified cells are not beta cells (beta islet cells) or beta cell precursors.

An aspect of this invention is the administration of genetically modified cells (e.g., human thyroid cells, hepatocytes, endothelial cells, epithelial cells, cortisol/aldosterone-producing cells, or precursors, e.g. stem cells) comprising a genetic modification or transgene encoding a fugetactic agent (e.g., CXCL12) to subjects in need thereof to treat autoimmune disease in the subject. In addition, the expression of a sufficient amount of a fugetactic agent protects against the risk of destruction of the genetically modified cells by mononuclear immune cell infiltration. The genetically modified human cells may be autologous or allogeneic. In an embodiment of this invention, the genetically modified cells are autologous cells derived from the patient suffering from an autoimmune disease, e.g., Graves' Disease, Crohn's Disease, Addison's Disease, autoimmune hepatitis, Hashimoto's Thyroiditis, Reactive Arthritis, Giant-cell Arteritis (GCA), or celiac disease, for example stem cells. In an embodiment, the patient does not have type 1 diabetes or multiple sclerosis. In another embodiment, the genetically modified cells are allogeneic human cells.

Another aspect of this invention relates to genetically modified human cells that are capable of expressing a fugetactic effective amount of a fugetactic agent (e.g., CXCL12) so as to be resistant to immune destruction. The fugetactic agent (e.g., CXCL12) may be an endogenous agent, i.e., an agent expressed by the subject to be treated, or an exogenous agent, e.g., an agent from a non-autologous source or a modified fugetactic agent. In one embodiment, the gene encoding the fugetactic agent in the cells or precursors is modified to be over-expressed compared to the unmodified gene. Methods for modifying gene expression are known in the art, for example, site-directed gene editing to replace the endogenous promoter with a different promoter (e.g., a constitutive promoter, an inducible promoter, etc.). In one embodiment, a recombinant polynucleotide encoding the fugetactic agent is inserted into the genetically modified cells, such that the fugetactic agent is expressed from the recombinant polynucleotide. Methods for inserting recombinant genes into a cell (transduction, transfection, etc.) are well known in the art, as are methods for making vectors with recombinant polynucleotides for insertion in to cells.

In some embodiments, the fugetactic agent is a modified fugetactic agent. For example, the polypeptide sequence of the fugetactic agent may be modified to increase circulating half-life, to incorporate conservative amino acid changes, enhance binding to an extracellular matrix, improve activity of the agent, etc. Accordingly, genes encoding a modified fugetactic agent (e.g., CXCL12) can be modified such the gene has at least 95% sequence identity to the native gene and preferably 99% sequence identity to the native gene. Likewise, the amino acid sequence of the modified fugetactic agent (e.g., modified CXCL12) has a sequence identity to the native agent of at least 95% and preferably 99%.

In one embodiment, there is provided a human thyroid cell comprising a vector that itself comprises a nucleic acid sequence encoding human CXCL12 or modified CXCL12 wherein said thyroid cell is made resistant to human immune cells.

In one embodiment, there is provided a human hepatocyte cell comprising a vector that itself comprises a nucleic acid sequence encoding human CXCL12 or modified CXCL12 wherein said hepatocyte cell is made resistant to human immune cells.

In one embodiment, there is provided a human endothelial cell comprising a vector that itself comprises a nucleic acid sequence encoding human CXCL12 or modified CXCL12 wherein said endothelial cell is made resistant to human immune cells.

In one embodiment, there is provided a human epithelial cell comprising a vector that itself comprises a nucleic acid sequence encoding human CXCL12 or modified CXCL12 wherein said epithelial cell is made resistant to human immune cells.

In one embodiment, there is provided a human cortisol/aldosterone-producing cell comprising a vector that itself comprises a nucleic acid sequence encoding human CXCL12 or modified CXCL12 wherein said cortisol/aldosterone-producing cell is made resistant to human immune cells.

In one embodiment, there is provided a human cstem cell comprising a vector that itself comprises a nucleic acid sequence encoding human CXCL12 or modified CXCL12 wherein said stem cell is made resistant to human immune cells.

In one embodiment, the human mononuclear immune cells comprise NK cells, T cells and B cells. In one embodiment, the T cells comprise cytotoxic T cells.

In one embodiment, the genetically modified human thyroid cell expresses human CXCL12 at a fugetactic amount.

In one embodiment, the genetically modified human hepatocyte cell expresses human CXCL12 at a fugetactic amount.

In one embodiment, the genetically modified human endothelial cell expresses human CXCL12 at a fugetactic amount.

In one embodiment, the genetically modified human epithelial cell expresses human CXCL12 at a fugetactic amount.

In one embodiment, the genetically modified human cortisol/aldosterone-producing cell expresses human CXCL12 at a fugetactic amount.

In one embodiment, the human CXCL12 is CXCL12 alpha or CXCL12 beta.

In one embodiment, the human genetically modified cell comprises a genetically modified regulatory region upstream of an endogenous CXCL12 coding region wherein said cell is resistant to human immune cells. Preferably, the endogenous CXCL12 coding region regulatory region comprises a constitutive promoter. In some embodiments, the endogenous CXCL12 coding region regulatory region comprises an inducible promoter.

In one embodiment, the human genetically modified cell comprises a genetically modified regulatory region upstream of an endogenous CXCL12 coding region wherein said cell is resistant to human immune cells is an autologous cell and, preferably, one obtained from a patient with an autoimmune disease.

In one embodiment, the human, genetically modified cell comprises the human gene for CXCL12. In one embodiment, the human genetically modified cell comprises the human gene selected from CXCL12 alpha and CXCL12 beta. In one embodiment, the human genetically modified cell comprises the human gene for CXCL12 beta. In one embodiment, the human genetically modified cell comprises the human gene for CXCL12 alpha.

In one embodiment, there is provided a human, genetically modified, senescent cell that comprises an expressible human CXCL12 gene wherein said cell expresses a fugetactic effective amount of CXCL12 so as to be resistant to human immune cells and further wherein said cell is senescent.

In one embodiment, the genetically modified cells or precursors described herein are obtained by:

(a) obtaining a population of human progenitor cells or human pluripotent stem cells from a human subject;

(b) genetically modifying the population of cells to express the fugetactic agent at a fugetactic amount to make genetically modified progenitor cells; and

(c) differentiating the genetically modified progenitor cells or pluripotent stem cells. In an embodiment, the genetically modified progenitor cells or pluripotent stem cells are differentiated into one or more cell types from the organ or tissue that is targeted by the autoimmune disease.

In one embodiment, the fugetactic agent is a cytokine, a chemokine, a CXCR4-binding antibody, a CXCR4 ligand, a CXCR5-binding antibody, or a CXCR5 ligand.

In one embodiment, the fugetactic agent is CXCL12.

In one aspect is provided a method for promoting survival of thyroid cells or precursors in a biological sample comprising immune cells by modifying thyroid cells or precursors to express a fugetactic agent at a level sufficient to inhibit or block immune cells from killing said thyroid cell.

In one aspect is provided a method for promoting survival of hepatocyte cells or precursors in a biological sample comprising immune cells by modifying hepatocyte cells or precursors to express a fugetactic agent at a level sufficient to inhibit or block immune cells from killing said hepatocyte cell.

In one aspect is provided a method for promoting survival of endothelial cells or precursors in a biological sample comprising immune cells by modifying endothelial cells or precursors to express a fugetactic agent at a level sufficient to inhibit or block immune cells from killing said endothelial cell.

In one aspect is provided a method for promoting survival of epithelial cells or precursors in a biological sample comprising immune cells by modifying epithelial cells or precursors to express a fugetactic agent at a level sufficient to inhibit or block immune cells from killing said epithelial cell.

In one aspect is provided a method for promoting survival of cortisol/aldosterone-producing cells or precursors in a biological sample comprising immune cells by modifying cortisol/aldosterone-producing cells or precursors to express a fugetactic agent at a level sufficient to inhibit or block immune cells from killing said cortisol/aldosterone-producing cell.

### DETAILED DESCRIPTION OF THE INVENTION

This invention provides for human cells that are genetically modified and/or comprise a transgene encoding a human fugetactic agent (e.g., CXCL12) or have been genetically modified to express or overexpress an endogenous (human) fugetactic agent (e.g., CXCL12) in fugetactic amounts. In a preferred embodiment, the genetically modified cells described herein are further modified to be senescent. In another of its method aspects, the cells are modified or treated so as to express an effective amount of a fugetactic agent (e.g., CXCL12) so as to inhibit immune destruction of the genetically modified cells and to reduce auto-inflammatory response.

Prior to disclosing this invention in further detail, the following terms will first be defined. If a term is not defined, it has its generally accepted scientific meaning as understood in the art.

The term “fugetaxis” or “fugetactic” refers to the ability of an agent to repel (or chemorepel) an eukaryotic cell with migratory capacity. A fugetactic amount of CXCL12 (or other fugetactic agent) expressed by a cell is an amount sufficient to block or inhibit immune cell migration towards the cell or in some aspects repel the immune cells from the cell.

The term “human immune cell” is used interchangeably with the term “human mononuclear immune cell” and includes NK cells, T cells, and B cells.

The term “immune cell-resistant” or “stealth to the immune system” indicates that the cell expresses an amount of fugetatic agent that is sufficient to block or inhibit immune cell migration towards the cell or in some aspects repel the immune cells from the cell. In a preferred embodiment, such blockage or inhibition is measured by the extent of cell death after exposure of the genetically modified cells of this invention to human mononuclear immune cells (e.g., PBMCs). Cell death can be assessed by release of lactate dehydrogenase (LDH) from cells that have undergone lysis. Preferably, immune cell resistant cells of this invention can be assessed by cells that evidence less than 50% of the LDH levels relative to control at a ratio of about 30:1 immune cells to cells of this invention over a two day period of incubation. More preferably, the genetically modified cells evidence less than 60% of the LDH level relative to control; and even more preferably, less than 75% of the LDH level relative to control; and most preferably, less than 95% of the LDH level relative to control. The procedure for assessing LDH levels is set forth in example 2 herein.

A fugetactic agent is an agent that has fugetactic activity. Fugetactic agents may include, without limitation, CXCL12.

The term “effector T-cell” refers to a differentiated T-cell capable of mounting a specific immune response by releasing cytokines.

The term “regulatory T-cell” refers to a T-cell that reduces or suppresses the immune response of B-cells or of other T-cells to an antigen.

The terms “CXCL12” or “SDF-1 polypeptide” refer to cytokines well-known in the art (see, for example, Table 1). In an embodiment, the terms refer to a protein or fragment thereof that binds a CXCL12 specific antibody and that has chemotaxis or fugetaxis activity. Chemotaxis or fugetaxis activity is determined by assaying the direction of T cell migration (e.g., toward or away from an agent of interest). See, e.g., Poznansky et al., Nature Medicine 2000, 6:543-8; N. Papeta et al., “Long-term survival of transplanted allogeneic cells engineered to express a T Cell chemorepellent,” Transplantation 2007, 83(2), 174-183. “Fugetaxis” or “Fugetactic migration” is the movement of a migratory cell away from an agent source (i.e., towards a lower concentration of agent). It is understood that the term “CXCL12” refers to all known isoforms thereof including the alpha, beta, gamma, delta, epsilon, phi and theta isoforms. Preferred CXCL12 isoforms are the alpha and beta. CXCL12 is known to induce angiogenesis.

The term “autoimmune disease” as used herein refers to refers to a disease or condition in which a subject's immune system has an aberrant immune response against a substance that does not normally elicit an immune response in a healthy subject. In particular, the autoimmune disease is characterized by immune cell attack on a particular tissue or organ. Non-limiting examples of autoimmune diseases include Graves' Disease, Crohn's Disease, Addison's Disease, autoimmune hepatitis, Hashimoto's Thyroiditis, Reactive Arthritis, Giant-cell Arteritis (GCA), or celiac disease. In an embodiment, the autoimmune disease is not type 1 diabetes. In an embodiment, the autoimmune disease is not multiple sclerosis.

As used herein, the term “thyroid cell or precursor” includes any cell that produces thyroid hormones thyroxine and triiodothyronine, or precursor of such a cell.

As used herein, the term “hepatocyte cell or precursor” includes any cell that produces bile slats, cholesterol, and/or phospholipids, or precursor of such a cell.

As used herein, the term “cortisol/aldosterone-producing cell or precursor” includes any cell that produces cortisol or aldosterone, or precursor of such a cell.

A “subject” or “patient” refers to a mammal, preferably to a human subject.

A “subject in need thereof” or “patient in need thereof” is a subject having an autoimmune disease.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The term “about” when used before a numerical designation, e.g., temperature, time, amount, concentration, and such other, including a range, indicates approximations which may vary by (+) or (−) 10%, 5%, 1%, or any subrange or subvalue there between. Other definitions appear in context throughout this disclosure.

An aspect of this invention are genetically modified cells, e.g., human thyroid cells, hepatocytes, endothelial cells, epithelial cells, cortisol/aldosterone-producing cells, or precursors of any thereof, comprising a nucleic acid encoding a fugetactic agent (e.g., CXCL12) in operable linkage with a promoter, such that the fugetactic agent (e.g., CXCL12) is expressed at a fugetactic level in the microenvironment of the cell. The promoter may be a promoter endogenous to the cells or heterologous to, but functional, in the cells. Preferably, the nucleic acid encoding the fugetactic agent (e.g., CXCL12) is endogenous to the subject being treated with the genetically modified cells (e.g., human fugetactic agent gene in a human patient).

In one embodiment, the genetically modified cell is autologous to the subject to be treated (and/or to the immune cells). In one embodiment, the genetically modified cell is allogenic to the subject to be treated (and/or to the immune cells). In one embodiment, the allogeneic cell is derived from a healthy donor.

An aspect of this invention are human cells comprising a genetically modified endogenous human gene encoding a fugetactic agent (e.g., CXCL12) wherein the gene is modified to comprise a heterologous promoter in operable linkage with the fugetactic agent—encoding sequence, such that the fugetactic agent is expressed from the endogenous gene at a fugetactic level in the cell microenvironment. The promoter may be introduced into the cells to be in operable linkage with the fugetactic agent-encoding sequence using genome editing techniques known in the art. It is well known that CXCL12 has several isoforms including the alpha, beta, gamma, and theta. In a preferred embodiment, the isoform employed is CXCL12 beta.

The genetically modified cells as described herein can be used to treat an autoimmune disease, in particular an autoimmune disease that is characterized by immune cell attack (immune cell-mediated cell death) of a particular organ or tissue (e.g., thyroid gland, adrenal gland, liver/hepatocytes, etc.). In embodiments, the genetically modified human cells or precursors described herein exhibit one or more activities exhibited by cells in the targeted organ or tissue (e.g., expression of one or more molecules that is normally produced by the organ or tissue, or cells therein).

In general, this invention provides for cells (e.g., thyroid cells, hepatocytes, endothelial cells, epithelial cells, cortisol/aldosterone-producing cells, or precursors), and preferably human cells, that express a fugetactic agent (e.g., CXCL12) at a level sufficient to block or inhibit migration of immune cells (e.g., human immune cells) to the genetically modified cells, and/or sufficient to repel immune cells. The terms immune cells and mononuclear cells (T-cells, B-cells, and NK cells) may be used interchangeably herein. The ability of a fugetactic agent (e.g., CXCL12) polypeptide to repel immune cells (e.g., effector T-cells) can be assessed in vitro, using a boyden chamber assay. See, e.g., as previously described in Poznansky et al., Journal of Clinical Investigation, 109, 1101 (2002). Alternatively, the viability of genetically modified human cells is assessed by combining such cells with human PBMC. The rate of cell death can be evaluated by measuring one or more cell death markers over time. One such marker commonly used is lactate dehydrogenase (LDH) that is released during cell necrosis.

Without wishing to be bound by any theory, Applicant contemplates that in an aspect of this invention the amount of fugetactic agent (e.g., CXCL12) produced by the genetically cell is sufficient to provide a fugetactic effect in the cell microenvironment, but is not produced in an amount sufficient to raise the systemic levels of the agent and upset the balance between the beneficial effects of the agent in one process while producing deleterious consequences in another. In addition, CXCL12 is known to induce angiogenesis when bound to its receptor CXCR4. Again, without being bound by any theory, it is contemplated that, in embodiments, the microenvironment of the implanted genetically modified cells expressing CXCL12 will induce an angiogenic response that enhance the survivability of the implanted cells.

The fugetactic effective amount of a fugetactic agent (e.g., CXCL12) is any amount sufficient to block immune cell from killing the genetically modified cell. For example a fugetactic effective amount of fugetactic agent (e.g., CXCL12) in the genetically modified cell microenvironment may be at least about 100 ng/mL, and preferably at least 100 nM. In some embodiments, the amount of fugetactic agent (e.g., CXCL12) in the genetically modified cell microenvironment is at least about 1000 ng/mL. For example, the following specific ranges that are suitable for this invention: from about 100 nM to about 200 nM, from about 100 nM to about 300 nM, from about 100 nM to about 400 nM, from about 100 nM to about 500 nM, from about 100 nM to about 600 nM, from about 100 nM to about 700 nM, from about 100 nM to about 800 nM, from about 100 nM to about 900 nM, or from about 100 nM to about 1 μM.

In embodiments, the fugetactic effective amount of fugetactic agent (e.g., CXCL12) in the genetically modified cell microenvironment ranges from 20 ng/mL to about 5 μg/mL. In embodiments, the fugetactic effective amount ranges from 20 ng/mL to about 1 μg/mL. In embodiments, the amount of the fugetactic agent (e.g., CXCL12) in the cell microenvironment is a fugetactic sufficient amount that ranges from about 100 ng/mL to about 500 ng/mL, from about 500 ng/mL to 5 μg/mL, about 800 ng/mL to about 5 μg/mL, or from about 1000 ng/mL to about 5000 ng/mL. Without wishing to be bound by theory, it is contemplated that when genetically modified and non-genetically modified cells are used together, the genetically modified cells can express sufficient amounts of the fugetactic agent such that the microenvironment creating the fugetactic effect extends to adjacent to non-genetically modified cells. The fugetactic effective amount of fugetactic agent (e.g., CXCL12) in the genetically modified cell microenvironment may be any value or subrange within the recited ranges, including endpoints.

CXCL12 polypeptides are known in the art. See, e.g., Poznansky et al., Nature Medicine 2000, 6:543-8 and US Patent Publ. No. 20170246250 both of which are incorporated herein by reference in their entirety. The terms CXCL12 and SDF-1 may be used interchangeably. Exemplary CXCL12/SDF1 Isoforms are provided in Table I of US Publ. 20170246250. Exemplary CXCL12/SDF1 Isoforms are also provided in Table 1 (below):

TABLE 1
HUMAN CXCL12/SDF1 ISOFORMS

| | | Accession | |
|---|---|---|---|
| | Accession | Number | |
| Name | Number | Versions | Sequence |
| SDF-1 | NP_954637 | NP_954637.1 | MNAKVVVVLV LVLTALCLSD |
| Alpha | | GI:40316924 | GKPVSLSYRC PCRFFESHVA |
| | | | RANVKHLKIL NTPNCALQIV |
| | | | ARLKNNNRQV CIDPKLKWIQ |
| | | | EYLEKALNK (SEQ ID NO: 1) |
| SDF-1 | P48061 | P48061.1 | MNAKVVVVLV LVLTALCLSD |
| Beta | | GI:1352728 | GKPVSLSYRC PCRFFESHVA |
| | | | RANVKHLKIL NTPNCALQIV |
| | | | ARLKNNNRQV CIDPKLKWIQ |
| | | | EYLEKALNKR FKM |
| | | | (SEQ ID NO: 2) |
| SDF-1 | NP_ | NP_001029058.1 | MNAKVVVVLV LVLTALCLSD |
| Gamma | 001029058 | GI:76563933 | GKPVSLSYRC PCRFFESHVA |
| | | | RANVKHLKIL NTPNCALQIV |
| | | | ARLKNNNRQV CIDPKLKWIQ |
| | | | EYLEKALNKG RREEKVGKKE |
| | | | KIGKKKRQKK RKAAQKRKN (SEQ |
| | | | ID NO: 3) |
| SDF-1 | | Yu et al. | MNAKVVVVLV LVLTALCLSD |
| Delta | | Identification | GKPVSLSYRC PCRFFESHVA |
| | | and expression | RANVKHLKIL NTPNCALQIV |
| | | of novel | ARLKNNNRQV CIDPKLKWIQ |
| | | isoforms of | EYLEKALNNL ISAAPAGKRV |
| | | human stromal | IAGARALHPS PPRACPTARA |
| | | cell-derived | LCEIRLWPPP EWSWPSPGDV (SEQ |
| | | factor 1. Gene | ID NO: 4) |
| | | (2006) vol. 374 | |
| | | pp. 174-9 | |
| SDF-1 | | Yu et al. | MNAKVVVVLV LVLTALCLSD |
| Epsilon | | Identification | GKPVSLSYRC PCRFFESHVA |
| | | and expression | RANVKHLKIL NTPNCALQIV |
| | | of novel | ARLKNNNRQV CIDPKLKWIQ |
| | | isoforms of | EYLEKALNNC (SEQ ID NO: 5) |
| | | human stromal | |
| | | cell-derived | |
| | | factor 1. Gene | |
| | | (2006) vol. 374 | |
| | | pp. 174-9 | |
| SDF-1 | | Yu et al. | MNAKVVVVLV LVLTALCLSD |
| Phi | | Identification | GKPVSLSYRC PCRFFESHVA |
| | | and expression | RANVKHLKIL NTPNCALQIV |
| | | of novel | ARLKNNNRQV CIDPKLKWIQ |
| | | isoforms of | EYLEKALNKIWLYGNAETSR (SEQ |
| | | human stromal | ID NO: 6) |
| | | cell-derived | |
| | | factor 1. Gene | |
| | | (2006) vol. 374 | |
| | | pp. 174-9 | |

In one embodiment, a CXCL12 polypeptide has at least about 85%, 90%, 92%, 95%, 96%, 97%, 98%, 99%, or 100% amino acid sequence identity to NP 001029058 and has chemokine or fugetactic activity. In one embodiment, a CXCL12 polypeptide has at least about 85%, 90%, 92%, 95%, 96%, 97%, 98%, 99%, or 100% amino acid sequence identity to SEQ ID NO: 1, SEQ ID NO: 2, SEQ ID NO: 3, SEQ ID NO: 4, SEQ ID NO: 5, or SEQ ID NO: 6, and has chemokine or fugetactic activity. Such sequence identity is based on the replacement of a first amino acid with a known conservative second amino acid. Such conservative replacements are well established in the art and the testing of the resulting modified CXCL12 polypeptide for its fugetactic properties are well known in the art. See, for example, Poznansky, supra.

The genetically modified cells used in the methods described herein may be autologous or non-autologous. “Autologous” cells are cells from the same individual. “Allogeneic” cells are cells from a genetically similar but not identical a donor of the same species. Allogenic cells useful in the methods of this invention are preferably from a human subject. Allogenic cells useful in the methods of this invention maybe from a relative e.g., a sibling, a cousin, a parent, or a child, or a non-relative. Criteria for selecting an allogenic donor are well known in the art and include HLA protein expression, see, e.g., J. Tiercy, Haematologica, June 2016 101: 680-687, which is incorporated herein by reference in its entirety. Human allogeneic cells, including stem cells, are commercially available and autologous tcells can be produced, for example, by the methods described by Stratton et al, eNeuro 2017, or Bakhuraysah Stem Cell Res Ther. 2016; 7: 12, each of which is incorporated by reference in its entirety.

In an embodiment, the genetically modified human cells used in the methods of this invention are autologous cells that can be prepared by deriving differentiated cells (e.g., thyroid cells, hepatocytes, endothelial cells, epithelial cells, cortisol/aldosterone-producing cells, or precursors) from multipotent progenitor cells or pluripotent stem cells obtained from the patient by methods known in the art. These derived cells may comprise (e.g., be transfected, infected, etc. with) an expression vector comprising a nucleic acid sequence encoding the fugetactic agent (e.g., CXCL12).

A “stem cell” is a cell characterized by the ability of self-renewal through mitotic cell division and the potential to differentiate into a tissue or an organ. Among mammalian stem cells, embryonic stem cells (ES cells) and somatic (or adult) stem cells can be distinguished. Embryonic stem cells reside in the blastocyst and give rise to embryonic tissues, whereas somatic stem cells reside in adult tissues for the purpose of tissue regeneration and repair. Adult stem cells include without limitation: mesenchymal stem cells (which can differentiate into a variety of cell types including osteoblasts, chondrocytes, myoctyes, and adipocytes), hematopoietic stem cells (which give rise to other blood cells), dental pulp stem cells, and endothelial stem cells. A “neural stem cell” or “NSC” refers to a stem cell capable to self-renew through mitotic cell division and to differentiate into a neural cell (e.g., glia cell, neuron, astrocyte, oligodendrocyte). An “induced pluripotent stem cell” or “iPSC” or “iPS” refers to a skin or blood cell that has been reprogrammed back into an embryonic-like pluripotent state.

Alternatively, the genetically modified cells used in the methods of this invention may be prepared by isolating differentiated or partially-differentiated cells from the subject in need thereof. These isolated cells may comprise (e.g., be transfected, infected, etc. with) an expression vector comprising a nucleic acid sequence encoding the fugetactic agent (e.g., CXCL12). Alternatively, the cell may be genetically modified to express the endogenous fugetactic agent (e.g., CXCL12) gene such that it constitutively produces a fugetactic effective amount of the fugetactic agent (e.g., CXCL12).

In an embodiment of this invention the genetically modified cells comprise (e.g., are transfected, infected, etc. with) an expression vector comprising a nucleic acid molecule that encodes the fugetactic agent (e.g., CXCL12), said nucleic acid molecule being in operable linkage with a promoter suitable for expression in the cell. The vector may integrate into the genome of the cell or it may exist episomally and not integrate into the genome. In embodiments, multiple vectors and/or integration sites are inserted into the cell in order to achieve expression of a fugetactic amount of the fugetactic agent (e.g., CXCL12).

The genetically modified cells of the invention may also be prepared from an adult stem cell by isolating adult stem cells from the subject, culturing the stem cells under appropriate conditions to expand the population and to induce differentiation into the desired cell type (e.g., thyroid cells, hepatocytes, endothelial cells, epithelial cells, cortisol/aldosterone-producing cells, or precursors of each thereof). The cells may be modified to express fugetactic effective amounts of the fugetactic agent (e.g., CXCL12) by introducing into the cells an expression vector encoding fugetactic amounts of the fugetactic agent (e.g., CXCL12) or by editing the genome to express fugetactic amounts of the fugetactic agent (e.g., CXCL12). The vector may be introduced into the stem cells prior to differentiation, or the genome of the stem cells may be edited to contain the heterologous promoter. Alternatively, the vector may be introduced into the resulting differentiated (or partially differentiated) cells or the genome of the resulting differentiated (or partially differentiated) cells may be edited to contain the heterologous promoter.

The genetically modified cells of the invention may also be prepared by generating induced pluripotent stem (iPS) cells from somatic cells, e.g., thyroid cells, hepatocytes, endothelial cells, epithelial cells, cortisol/aldosterone-producing cells, or precursors, fibroblasts or keratinocytes, of a subject; treating the iPS cells to induce differentiation into the desired cells or precursors; and introducing into the differentiated cells or precursors an expression vector comprising a nucleic acid sequence encoding the fugetactic agent (e.g., CXCL12).

The genetically modified cells of the invention may also be prepared by preparing induced pluripotent stem (iPS) cells generated from somatic cells of a subject; introducing into the iPS cells an expression vector comprising a nucleic acid sequence encoding the fugetactic agent (e.g., CXCL12); and treating the iPS cells, before or after introduction of the genetic modification, to induce differentiation, e.g. into thyroid cells, hepatocytes, endothelial cells, epithelial cells, cortisol/aldosterone-producing cells, or precursors.

The genetically modified cells of this invention may also be generated by obtaining progenitor cells or progenitor-like cells, e.g., hematopoietic stem cells (HSCs), introducing a vector comprising a nucleic acid sequence encoding the fugetactic agent (e.g., CXCL12) into the cells, and treating the cells either before or after introducing the vector to induce differentiation, e.g. into thyroid cells, hepatocytes, endothelial cells, epithelial cells, cortisol/aldosterone-producing cells, or precursors, by methods known in the art. The progenitor cell and progenitor-like cells may be autologous or non-autologous, e.g., allogeneic, to the subject treated with the genetically modified cells.

Any suitable somatic cell from a subject may be reprogrammed into an iPS cell by methods known in the art, see e.g., Yu et al. (2007). Induced pluripotent stem cell lines derived from human somatic cells. Science 318, 1917-1920; Takahashi and Yamanaka, 2006, Cell 126(4):663-676; Wernig et al., 2007, Nature 448:7151; Okita et al., 2007 Nature 448:7151; Maherali et al., 2007 Cell Stem Cell 1:55-70; Lowry et al., 2008 PNAS 105:2883-2888; Park et al., 2008 Nature 451:141-146.; Takahashi et al., 2007 Cell 131, 861-872; U.S. Pat. Nos. 8,546,140; 7,033,831 and; 8,268,620. The iPS cells may be differentiated into the desired cells or precursors using methods known in the art, see e.g. Amabile et al, 2013 Blood 121:1255-1264; Chou et al, 2013 Molecular Therapy 21:1292-1293.

Preferably the fugetactic agent-encoding sequence is in operable linkage with a regulatory region that is suitable for expression in the desired cell type, e.g., a thyroid cell, hepatocyte, endothelial cell, epithelial cell, cortisol/aldosterone-producing cell, or precursor cell. Suitable regulatory regions are known in the art, and include promoters such as, e.g., mammalian promoters including, e.g., hypoxanthine phosphoribosyl transferase (HPTR), adenosine deaminase, pyruvate kinase, β-actin promoter, muscle creatine kinase promoter, and human elongation factor promoter (EF1a), a GAPDH promoter, an actin promoter, and an ubiquitin promoter and viral promoters including SV40 early promoter, SV40 late promoter, metallothionein promoter, murine mammary tumor virus promoter, Rous sarcoma virus promoter, polyhedrin promoter, human immunodeficiency virus (HIV) promoters, cytomegalovirus (CMV) promoters, adenoviral promoters, adeno-associated viral promoters, or the thymidine kinase promoter of herpes simplex virus. Other relevant promoters, e.g., viral and eukaryotic promoters, are also well known in the art (see e.g., in Sambrook and Russell (Molecular Cloning: a laboratory manual, Cold Spring Harbor Laboratory Press). The regulatory region in operable linkage with the fugetactic agent-encoding sequence may be any constitutive promoter suitable for expression in the subject's cells.

The genetically modified cells expressing the fugetactic agent (e.g., CXCL12) of this invention, whether autologous or non-autologous, e.g., allogeneic, may be administered to a subject in need thereof by any means known in the art for administering such cells. The genetically modified cells of this invention may be administered in an amount sufficient to provide levels of HSCs able to alleviate at least some of the symptoms associated with autoreactive T and B cells and immune attack on the organ or tissues targeted in the autoimmune disease being treated.

Another aspect of the invention is a method of treating autoimmune diseases in a subject in need thereof, comprising the steps of: (a) obtaining or deriving cells, e.g., thyroid cells, hepatocytes, endothelial cells, epithelial cells, cortisol/aldosterone-producing cells, or precursors or stem cells from the subject; (b) introducing a suitable expression vector encoding the fugetactic agent (e.g., CXCL12) into the cells to form autologous genetically modified cells expressing the introduced the fugetactic agent (e.g., CXCL12); and (c) transplanting the autologous genetically modified cells into the subject. Optionally, the stem cells or precursor cells are differentiated or partially differentiated prior to administration to the subject.

Many vectors useful for transferring exogenous genes into mammalian cells, e.g., thyroid cells, hepatocytes, endothelial cells, epithelial cells, cortisol/aldosterone-producing cells, or precursors, including vectors that integrate into the genome and vectors that do not integrate into the genome but exist as episomes, and methods for introducing such vectors into cells are available and known in the art. For example, retroviral vectors, lentiviral vectors, adenoviral vectors, adeno-associated (AAV)—based vectors and EBV-based vectors may be used. See, e.g., US 20110280842, Narayanavari and Izsvák, Cell Gene Therapy Insights 2017; 3(2), 131-158; Hardee et al., Genes 2107, 8, 65; Tipanee et al., Bioscience Reports (2017) 37, and Chira et al. Oncotarget, Vo. 6, No. 31, pages 30675-30703.

Another aspect of the invention is a method for promoting survival of cells targeted in autoimmune disease, e.g., thyroid cells, hepatocytes, endothelial cells, epithelial cells, cortisol/aldosterone-producing cells, or precursors (e.g. stem cells), in a biological sample comprising immune cells comprising introducing an expression vector encoding the fugetactic agent (e.g., CXCL12) into the cells, or by editing the genome of the cells such that the cells express fugetactic amounts of the fugetactic agent (e.g., CXCL12). In an aspect of this invention the fugetactic agent (e.g., CXCL12) is expressed by the cells at a level sufficient to block or inhibit migration of immune cells, e.g. T-cells, B-cells, and/or NK cells, to the cells. In an aspect of this invention the fugetactic agent (e.g., CXCL12) is expressed by the cells at a level sufficient to repel the immune cells from the cells. In an aspect of this invention the genetically modified cells are in a subject, e.g., a human subject having an autoimmune disease. In one embodiment, the cells are autologous from the subject.

Methods for the delivery of viral vectors and non-viral vectors to mammalian cells are well known in the art and include, e.g., lipofection, microinjection, ballistics, virosomes, liposomes, immunoliposomes, polycation or lipid-nucleic acid conjugates, naked DNA, artificial virions, and agent-enhanced uptake of DNA. Lipofection reagents are sold commercially (e.g., Transfectam™ and Lipofectin™). Cationic and neutral lipids suitable for efficient receptor-recognition lipofection of polynucleotides are known. Nucleic acid can be delivered to cells (ex vivo administration) or to target tissues (in vivo administration). The preparation of lipid:nucleic acid complexes, including targeted liposomes such as immunolipid complexes, is well known to those of skill in the art. Recombination mediated systems can be used to introduce the vectors into the cells. Such recombination methods include, e.g., use of site specific recombinases like Cre, Flp or PHIC31 (see e.g. Oumard et al., Cytotechnology (2006) 50: 93-108) which can mediate directed insertion of transgenes or other genetic modifications.

Vectors suitable for use in this invention include expression vectors comprising a nucleic acid encoding a fugetactic agent (e.g., CXCL12) in operable linkage with a promoter to direct transcription. Suitable promoters are well known in the art and described, e.g., in Sambrook and Russell (Molecular Cloning: a laboratory manual, Cold Spring Harbor Laboratory Press). The promoter used to direct expression of the fugetactic agent (e.g., CXCL12) may be, e.g., example, SV40 early promoter, SV40 late promoter, metallothionein promoter, murine mammary tumor virus promoter, Rous sarcoma virus promoter, or other promoters shown to be effective for expression in mammalian cells.

Vectors useful in the methods of this invention include, e.g., SV40 vectors, papilloma virus vectors, Epstein-Barr virus vectors, retroviral vectors, and lentiviral vectors.

The vectors used in this invention may comprise regulatory elements from eukaryotic viruses, e.g., SV40, papilloma virus, and Epstein-Barr virus, including e.g., signals for efficient polyadenylation of the transcript, transcriptional termination, ribosome binding, and/or translation termination. Additional elements of the vectors may include, e.g., enhancers, and heterologous spliced intronic signals.

In an embodiment of this invention, the genome of the cell may be genetically modified to increase the expression levels of an endogenous fugetactic agent (e.g., CXCL12) gene. Such increased expression may be achieved by introducing a heterologous promoter in operable linkage with the endogenous fugetactic agent (e.g., CXCL12) gene or by altering the endogenous fugetactic agent (e.g., CXCL12) promoter such that the cell expresses a fugetactic level of fugetactic agent (e.g., CXCL12). Such increased expression may be achieved by introducing a promoter into the genome of the cell such that it is in operable linkage with the endogenous fugetactic agent-encoding sequence and thereby expresses or overexpresses the fugetactic agent in a fugetactic amount.

Gene editing technologies for modifying the genome are well known in the art and include e.g., CRISPR/CAS 9, Piggybac, Sleeping Beauty genome editing systems, (see for example, Zhang et al. Molecular Therapy Nucleic Acids, Vol 9, December 2017, page 230-241; systems (see e.g., Cong et al., Science. 2013; 339(6121): 819-23; Mali et al., Science. 2013; 339(6121): 823-6; Gonzalez et al., Cell Stem Cell. 2014; 15(2): 215-26); He et al., Nucleic Acids Res. 2016; 44(9); Hsu et al., Cell. 2014; 157(6): 1262-78.), zinc finger nuclease-based systems (see e.g., Porteus and Carroll, Nat Biotechnol. 2005; 23(8): 967-73; Urnov et al., Nat Rev Genet. 2010; 11(9): 636-46), TALEN-based systems (transcription activator-like effector nucleases)(see e.g., Cermak et al., Nucleic Acids Res. 2011; 39(12); Hockemeyer et al., Nat Biotechnol. 2011; 29(8): 731-4; Joung and Sander J D, Nat Rev Mol Cell Biol. 2013; 14(1): 49-55; Miller et al., Nat Biotechnol. 2011; 29(2): 143-8, and Reyon et al., Nat Biotechnol. 2012; 30(5): 460-5).

In one embodiment, the genetically modified cells described herein are treated with an agent that renders the cells viable and capable of controlling immune function in a patient but unable to replicate (i.e., induced cellular senescence). One such agent is Mitomycin C that is a known DNA cross-linking agent. Upon treatment, the DNA in these cells is cross-linked thereby rendering impossible the formation of single stranded DNA necessary for replication. Such a treatment prevents the cells, especially those generated from stem cells, from dividing such that if the cell morphs into a cancer cell it cannot divide. Other known agents capable of inducing cellular senescence include those recited by Petrova, et al., “Small Molecule Compounds that Induce Cellular Senescence” Aging Cell, 15(6):999-1017 (2016) which reference is incorporated herein in its entirety. Such agents include, by way of example only, agents that cause telomere dysfunction due to replication-associated telomere shortening, subcytoxic stresses such as exposure to UV, gamma irradiation, hydrogen peroxide, and hypoxia. The specific means by which the cells or precursors of this invention are rendered non-replicative is not critical provided that these cells can be implanted without risk of cellular division.

In one embodiment, the genetically modified cells described herein comprise a conditionally-expressible gene that acts as a “kill switch” for the cells. For example, expression of the conditionally-expressible (e.g., inducible) gene may result in apoptosis, necrosis, or senescence of the cell. Genes that cause apoptosis, necrosis, or senescence of cells are known in the art, including, without limitation, Dicer, caspase 9, caspase 3, DNA Fragmentation Factor, and variants thereof. See, e.g., U.S. Pub. No. 2013/0323834; U.S. Pat. Nos. 7,638,331 and 6,165,737; each of which is incorporated herein by reference in its entirety. Inducible promoters are well known in the art, including, without limitation, the radiation-inducible promoters, e.g. the early growth response-1 gene (egr-1); heat-inducible promoters, e.g., gadd 153 and hsp70; Metallothionein (MT) and 1,24-vitamin D(3)(OH)(2) dehydroxylase (VDH) promoters; and the like. See, e.g., Schmidt et al., Eur Arch Otorhinolaryngol. 2004 April; 261(4):208-15; Ito et al., Cancer Gene Therapy, Vol 8, No 9, 2001: pp 649-654; U.S. Pat. No. 7,041,653; PCT Pub. No. WO 1992/011033; Itai et al., Clin Exp Dermatol. 2001 September; 26(6):531-5.

Another aspect of the invention is a method of treating an autoimmune disease in a subject, comprising administering to the subject in need thereof the cells or precursors (e.g., stem cells) described herein, wherein the cells or precursors express a fugetactic agent (e.g., CXCL12) in a fugetactic amount. The cells or precursors may be autologous cells or precursors or non-autologous cells or precursors, e.g. allogeneic cells or precursors, and may harbor a vector expressing the fugetactic agent, which vector may be integrated into the cell genome or exist episomally. In an embodiment of this invention the genetically modified cells or precursors may be a genetically modified to overexpress endogenous fugetactic agent (e.g., CXCL12) at a fugetactic level.

Without being bound by theory, it is believed that administration to a patient of a stem cell or precursor that expresses a fugetactic agent in an amount sufficient to render the cell resistant to immune cells will result in migration of the cell to an area of the patient in need of, e.g. to an organ or tissue that is attacked by the immune cells in the autoimmune disease being treated. It is further believed that the cell will differentiate into a cell appropriate for that organ or tissue and contribute to proper function of the organ or tissue.

Methods of introducing the genetically modified cells described herein into individuals are well known to those of skills in the art and include, but are not limited to, injection, intravenous, intraportal, or parenteral administration. Single, multiple, continuous or intermittent administration can be effected. See e.g., Schuetz and Markmann, Curr Transplant Rep. 2016 September; 3(3): 254-263.

Pharmaceutically acceptable carriers include sterile aqueous solutions or dispersions and sterile powders for the extemporaneous preparation of sterile injectable solutions or dispersion. The use of media and agents for pharmaceutically active substances, including cells, is well known in the art. A typical pharmaceutical composition for intravenous infusion of the cells or precursors could be made up to contain 250 ml of sterile Ringer's solution, and 100 mg of the combination. Actual methods for preparing parenterally administrable compounds will be known or apparent to those skilled in the art and are described in more detail in for example, Remington's Pharmaceutical Science, 17th ed., Mack Publishing Company, Easton, Pa. (1985), and the 18th and 19th editions thereof, which are incorporated herein by reference.

The genetically modified cells of the invention can be introduced into any of several different sites well known in the art, including but not limited to the pancreas, the abdominal cavity, the kidney, the liver, the portal vein or the spleen of the subject.

In addition, in order to avoid any possible transformation of the genetically modified cells into cancer cells that could result in the possibility of the patient developing a tumor, the genetically modified cells can be rendered senescent by contacting with known agents such as Mitomycin C, or exposure to subtoxic stress from ionizing radiation, hypoxia, hydrogen peroxide, etc. Cells derived from pluripotent stem cells typically undergo apoptosis during inappropriate cell division or due to immune cell clearance. The senescent genetically modified cells described herein are incapable of division thereby eliminating apoptotic triggers arising during cellular division. In addition, the senescent genetically modified cells described herein are immune cell resistant thereby providing protection against apoptosis induction due to immune cell clearance. Accordingly, it is contemplated that the genetically modified cells described herein will have a longer lifespan to a significantly longer lifespan than non-senescent genetically modified cells.

The genetically modified and optionally senescent modified cells may be transplanted into the subject via a graft. An ideal cell, e.g. thyroid cell, hepatocyte, endothelial cell, epithelial cell, cortisol/aldosterone-producing cell, or precursor cell, transplantation site would be one that supports the implantation, long-term function and survival of grafted cells in the subject and is easily accessible for maximal patient safety. Sites for implantation include the liver, intestinal, subdermal, and pancreatic sites.

The following abbreviations used herein have the following meanings and if abbreviations are not defined, they have their generally accepted scientific meaning. Amino acids are recited herein using their established one letter abbreviations.
FLAG=DYKDDDDK protein tag (SEQ ID NO: 9)
g/L=grams per liter
HRP=horseradish peroxidase
LDH=lactate dehydrogenase
iBLOT=Semi-dry protein transfer device (Invitrogen)
MES=2-(N-morpholino)ethanesulfonic acid
mL=milliliter
N/A=not applicable
nM=nanomolar
PBMC=peripheral blood mononuclear cells
PBS=phosphate buffered saline
TMB=3,3′,5,5′-Tetramethylbenzidine
μL=microliters
μg=micrograms
μg=times gravity

### EXAMPLES

### Example 1: Model Cells Will be Used to Assess Expression Levels of CXCL12-a and -b Isoforms

HEK293 cells will be transfected with 2 different isoforms of CXCL12 (alpha and beta) using commercially available plasmids for each isoform (plasmids available from GenScript). Transfected cells will be selected with 250 ug/mL of G418 (commercially available from ThermoFisher) and a stable pool for each isoform will be created. Cells will be allowed to condition a suitable medium for 3 days. Conditioned medium from the transfected HEK293 cells expressing CXCL12 alpha and CXCL12 beta will be diluted 1:1 with assay dilution buffer. Two separate pools will be established for each isoform and then the concentration of each isoform in solution will be obtained by absorption using a standardized concentration curve. This experiment will be repeated twice.

The results will show evidence that genetically modified model cells express CXCL12 beta at significantly higher levels than genetically modified model cells that express CXCL12 alpha.

### Example 2: Model Cells Will be Used to Assess Expression Levels of Other Isoforms of CXCL12

HEK293 cells will be transfected with 5 different isoforms of CXCL12 (alpha and beta) using commercially available plasmids for each isoform (plasmids available from GenScript). Transfected cells will be selected with 250 ug/mL of G418 (commercially available from ThermoFisher) and a stable pool for each isoform will be created. Cells will be allowed to condition in a suitable medium for 3 days. The conditioned medium will be separated in a 4-8% NuPage gel (commercially available from ThermoFisher) with IVIES buffer and transferred to nitrocellulose (iBLOT).

Expression levels will be detected with HRP labeled, anti-FLAG tag antibody/TMB chromogen (available from GenScript) on a Western Blot. The results will evidence that the gamma, delta and theta isoforms of CXCL12 have greater concentrations than the alpha or beta isoforms.

### Example 3: Preparation of Genetically Modified Thyroid Cells, Hepatocytes, Endothelial Cells, Epithelial Cells, Cortisol/Aldosterone-Producing Cells, or Precursors

Thyroid cells, hepatocytes, endothelial cells, epithelial cells, cortisol/aldosterone-producing cells, or precursors derived from human induced pluripotent stem cells will be purchased from Takara Bio USA, Inc. (Mountain View, Calif.) and cultured according to provided instructions.

Cells will be transduced with lentiviral vectors (pLenti-C-Myc-DDK, OriGene Technologies, Rockville, Md.) containing a human CXCL12 isotype (CXCL12a/SDF-1alpha or CXCL12b/SDF-1beta) or control. The lentiviral vectors will be used at a ratio of about 10:1 per cell. The sequences, including the tag (underlined) are provided below. Concentration of the CXCL12 isotype will be determined by ELISA (RayBioTech, Norcross, Ga.).

CXCL12a (aka SDF1a)
Accession No. NM_199168
SEQ ID NO.: 8
ATGAACGCCAAGGTCGTGGTCGTGCTGGTCCTCGT
GCTGACCGCGCTCTGCCTCAGCGACGGGAAGCCCG
TCAGCCTGAGCTACAGATGCCCATGCCGATTCTTC
GAAAGCCATGTTGCCAGAGCCAACGTCAAGCATCT
CAAAATTCTCAACACTCCAAACTGTGCCCTTCAGA
TTGTAGCCCGGCTGAAGAACAACAACAGACAAGTG
TGCATTGACCCGAAGCTAAAGTGGATTCAGGAGTA
CCTGGAGAAAGCTTTAAACAAGACGCGTACGCGGC
CGCTCGAGCAGAAACTCATCTCAGAAGAGGATCTG
GCAGCAAATGATATCCTGGATTACAAGGATGACGA
CGATAAGGTTTAA
CXCL12b (aka SDF1b)
Accession No. NM_000609
SEQ ID NO.: 7
ATGAACGCCAAGGTCGTGGTCGTGCTGGTCCTCGT
GCTGACCGCGCTCTGCCTCAGCGACGGGAAGCCCG
TCAGCCTGAGCTACAGATGCCCATGCCGATTCTTC
GAAAGCCATGTTGCCAGAGCCAACGTCAAGCATCT
CAAAATTCTCAACACTCCAAACTGTGCCCTTCAGA
TTGTAGCCCGGCTGAAGAACAACAACAGACAAGTG
TGCATTGACCCGAAGCTAAAGTGGATTCAGGAGTA
CCTGGAGAAAGCTTTAAACAAGAGGTTCAAGATGA
CGCGTACGCGGCCGCTCGAGCAGAAACTCATCTCA
GAAGAGGATCTGGCAGCAAATGATATCCTGGATTA
CAAGGATGACGACGATAAGGTTTAA

### Example 4: Genetically Modified Thyroid Cells, Hepatocytes, Endothelial Cells, Epithelial Cells, Cortisol/Aldosterone-Producing Cells, or Precursors Repel PBMCs

The genetically modified thyroid cells, hepatocytes, endothelial cells, epithelial cells, cortisol/aldosterone-producing cells, or precursors from Example 3 will be contacted with human peripheral blood mononuclear cells (PBMCs, Innovative Research, Novi, Mich.) at a ratio of 30:1 (PBMCs to cell). Briefly, PBMCs will be resuspended in culture medium, counted and adjusted to allow for a 30:1 PBMC:cell ratio with addition of 100 uL of PBMCs (to minimize dilution of the expressed CXCL12). Final volume will be 1.1 mL. Background controls of thyroid cells, hepatocytes, endothelial cells, epithelial cells, cortisol/aldosterone-producing cells, or precursors without PBMCs and PBMCs without thyroid cells, hepatocytes, endothelial cells, epithelial cells, cortisol/aldosterone-producing cells, or precursors will also be created. Immediately 150 uL of medium will be removed from each sample and centrifuged at 1200×g for 10 minutes. Supernatant will be removed and stored at 4° C. (time zero). Cells will be returned to the incubator and sampled in a similar way to the time zero sample at both 24 and 48 hours later.

Release of LDH will be tested at 24 and 48 hours after contact using Pierce LDH Cytotoxicity Assay Kit (Thermo Scientific) according to manufacturer's instructions. Increased LDH is an indicator of cytotoxicity (cell lysis).

These data indicate that expression of CXCL12 by thyroid cells, hepatocytes, endothelial cells, epithelial cells, cortisol/aldosterone-producing cells, or precursors protects the thyroid cells, hepatocytes, endothelial cells, epithelial cells, cortisol/aldosterone-producing cells, or precursors from immune cell attack thereby rendering them resistant. Thyroid cells, hepatocytes, endothelial cells, epithelial cells, cortisol/aldosterone-producing cells, or precursors expressing SDF1b/CXCL12b will show essentially no cytotoxicity in the presence of PBMCs.

### Example 5: Alternative Preparation of Genetically Modified Thyroid Cells, Hepatocytes, Endothelial Cells, Epithelial Cells, Cortisol/Aldosterone-Producing Cells, or Precursors

Thyroid cells, hepatocytes, endothelial cells, epithelial cells, cortisol/aldosterone-producing cells, or precursors isolated from a subject having an autoimmune disease will be transfected or infected in vitro with a retroviral expression vector encoding CXCL12 or a control retroviral vector that does not encode CXCL12. Genetically modified thyroid cells, hepatocytes, endothelial cells, epithelial cells, cortisol/aldosterone-producing cells, or precursors harboring the retroviral vector encoding CXCL12 will be assayed for expression of fugetactic amounts of CXCL12 using a Boyden chamber assay as previously described in Poznansky et al., Journal of Clinical Investigation, 109, 1101 (2002). It is expected that genetically modified thyroid cells, hepatocytes, endothelial cells, epithelial cells, cortisol/aldosterone-producing cells, or precursors expressing at least 100 nM CXCL12 will repel immune cells in this assay.

### Example 6: Effect of Forced Senescence of Genetically Modified Thyroid Cells, Hepatocytes, Endothelial Cells, Epithelial Cells, Cortisol/Aldosterone-Producing Cells, or Precursors on Genetically Modified Cytokine Expression

Thyroid cells, hepatocytes, endothelial cells, epithelial cells, cortisol/aldosterone-producing cells, or precursors will be prepared as described in Example 3. Expression levels of SDF1a/CXCL12a and SDF1b/CXCL12b will be assayed by ELISA before Mitomycin C (available from Santa Cruz Biotechnology) treatment to determine baseline expression (“Before”). Medium was replaced with fresh medium containing 10 ug/mL Mitomycin C—an agent known to induce senescence. Cells will be returned to the incubator for 2 hours. The mitomycin C containing medium will be removed by gentle pipetting. The cells will be washed with PBS twice. After the second wash, the cells will be fed fresh complete medium. SDF1a/CXCL12a or SDF1b/CXCL12b expression will be determined by ELISA assay. SDF1a/CXCL12a and SDF1b/CXCL12b expression is not affected by forced senescence of the genetically modified thyroid cells, hepatocytes, endothelial cells, epithelial cells, cortisol/aldosterone-producing cells, or precursors.

TABLE 2
CXCL12a and -b levels before and after Mitomycin C treatment

| | Before | |
|---|---|---|
| Cytokine | Mitomycin C | After Mitomycin C |
| CXCL12a | 97.5 nM | 100.5 nM |
| Pool 12 | | |
| CXCL12a | 98.2 nM | 99.4 nM |
| Pool 22 | | |
| CXCL12b | 806.2 nM | 789.3 nM |
| Pool 12 | | |
| CXCL12b | 757.1 nM | 788.0 nM |
| Pool 22 | | |

### Example 7: Effect of Forced Senescence of Genetically Modified Thyroid Cells, Hepatocytes, Endothelial Cells, Epithelial Cells, Cortisol/Aldosterone-Producing Cells, or Precursors on PBMC Challenge

Thyroid cells, hepatocytes, endothelial cells, epithelial cells, cortisol/aldosterone-producing cells, or precursors will be prepared as described in Example 3. Cells will be treated with Mitomycin C or control as described in Example 4. Cells will be contacted with PBMCs as described in Example 2.

LDH levels are not expected to be affected by forced senescence of the genetically modified thyroid cells, hepatocytes, endothelial cells, epithelial cells, cortisol/aldosterone-producing cells, or precursors.

### Example 8: In Vivo Evaluation of Genetically Modified Thyroid Cells, Hepatocytes, Endothelial Cells, Epithelial Cells, Cortisol/Aldosterone-Producing Cells, or Precursors

Humanized mice having a humanized immune system, see e.g., N. Walsh, “Humanized mouse models of clinical disease,” Annu Rev Pathol 2017, 12, 187-215; E. Yoshihara et al., will be administered either the genetically modified human thyroid cells, hepatocytes, endothelial cells, epithelial cells, cortisol/aldosterone-producing cells, or precursors expressing fugetactic amounts of CXCL12 or the control genetically modified human thyroid cells, hepatocytes, endothelial cells, epithelial cells, cortisol/aldosterone-producing cells, or precursors and the survival of the genetically modified thyroid cells, hepatocytes, endothelial cells, epithelial cells, cortisol/aldosterone-producing cells, or precursors in the mice will be assayed at various time points after the initial administration. It is contemplated that the genetically modified human thyroid cells, hepatocytes, endothelial cells, epithelial cells, cortisol/aldosterone-producing cells, or precursors expressing fugetactic amounts of CXCL12 will survive for longer periods than the control genetically modified human thyroid cells, hepatocytes, endothelial cells, epithelial cells, cortisol/aldosterone-producing cells, or precursors.

Humanized mice having a humanized immune system, see e.g., N. Walsh, “Humanized mouse models of clinical disease,” Annu Rev Pathol 2017, 12, 187-215; E. Yoshihara et al., will be administered either genetically modified human thyroid cells, hepatocytes, endothelial cells, epithelial cells, cortisol/aldosterone-producing cells, or precursors overexpressing CXCL12 from an endogenous CXCL12 gene, or control human thyroid cells, hepatocytes, endothelial cells, epithelial cells, cortisol/aldosterone-producing cells, or precursors and the production of the desired molecules, and survival of the thyroid cells, hepatocytes, endothelial cells, epithelial cells, cortisol/aldosterone-producing cells, or precursors in the mice will be assayed at various time points after the initial administration. It is contemplated that the genetically modified human thyroid cells, hepatocytes, endothelial cells, epithelial cells, cortisol/aldosterone-producing cells, or precursors overexpressing CXCL12 will survive for longer periods than the control human thyroid cells, hepatocytes, endothelial cells, epithelial cells, cortisol/aldosterone-producing cells, or precursors that were not genetically modified to overexpress CXCL12. It is also contemplated that mice receiving the genetically modified human thyroid cells, hepatocytes, endothelial cells, epithelial cells, cortisol/aldosterone-producing cells, or precursors overexpressing CXCL12 will also have higher amounts of one or more molecules expressed by the cells than mice receiving the control human thyroid cells, hepatocytes, endothelial cells, epithelial cells, cortisol/aldosterone-producing cells, or precursors and the higher levels will persist for longer periods of time as compared to the levels in mice administered the control human thyroid cells, hepatocytes, endothelial cells, epithelial cells, cortisol/aldosterone-producing cells, or precursors.

Optionally, the cells will be treated with an agent that cross-links the DNA within the cell to prevent cell division (e.g., Mitomycin C).

The foregoing description has been set forth merely to illustrate the invention and is not meant to be limiting. Since modifications of the described embodiments incorporating the spirit and the substance of the invention may occur to persons skilled in the art, the invention should be construed broadly to include all variations within the scope of the claims and equivalents thereof.

1. A human cell that expresses or overexpresses a human fugetactic agent in an amount sufficient to render said cell resistant to human immune cells, wherein said cell is a cell that is attacked by the immune system of a patient having an autoimmune disease, or a precursor of a cell that is attacked by the immune system of a patient having the autoimmune disease.

2. The human cell of claim 1, wherein the autoimmune disease is selected from Graves' Disease, Crohn's Disease, Addison's Disease, autoimmune hepatitis, Hashimoto's Thyroiditis, Reactive Arthritis, Giant-cell Arteritis (GCA), and celiac disease.

3. The human cell of claim 1 or 2, wherein the autoimmune disease is not type 1 diabetes or multiple sclerosis.

4. The human cell thereof of any one of the above claims, wherein the cell is a stem cell obtained from a subject with an autoimmune disease.

5. The human cell of any one of claims 1-3, wherein the cell is an allogenic stem cell.

6. The human cell of any one of claims 1-4, wherein said human immune cells comprise NK cells, cytotoxic T cells and/or B cells.

7. The human cell or precursor thereof of any one of claims 1-6, wherein said cell expresses human CXCL12 at a fugetactic amount.

8. The human cell or precursor thereof of claim 7, wherein said CXCL12 is selected from CXCL12 alpha and CXCL12 beta.

9. The human cell or precursor thereof of any one of the above claims, further comprising a conditionally-expressed gene that causes apoptosis in the cell.

10. A human cell or precursor thereof, comprising a genetically modified regulatory region upstream of an endogenous CXCL12 coding region wherein said cell is resistant to human immune cells, wherein said cell is a cell that is attacked by the immune system of a patient having an autoimmune disease, or a precursor of a cell that is attacked by the immune system of a patient having the autoimmune disease.

11. The human cell or precursor thereof of claim 10, wherein the cell is an autologous cell.

12. The human cell or precursor thereof of claim 11, wherein the cell is an autologous cell obtained or derived from a subject with an autoimmune disease.

13. The human cell or precursor thereof of claim 10, wherein the cell is an allogenic cell.

14. The human cell or precursor thereof of claim 13, wherein the cell is an allogeneic cell obtained or derived from a subject free of autoimmune disease.

15. The human cell or precursor thereof of any one of claims 10-14, wherein the genetically modified regulatory region is an exogenous constitutive, or inducible promoter.

16. The human cell or precursor thereof of any one of claims 10-15, wherein the cell expresses CXCL12 at a fugetactic amount.

17. The human cell or precursor thereof of claim 16, wherein said CXCL12 is selected from CXCL12 alpha and CXCL12 beta.

18. The human cell or precursor thereof of claim 17, wherein the cell expresses CXCL12 beta.

19. The human cell or precursor thereof of any one of claims 1-18, wherein the cell is incapable of cell division.

20. The human cell or precursor thereof of any one of claims 1-19, wherein the human CXCL12 is selected from CXCL12 alpha, CXCL12 beta, CXCL12 delta, and CXCL12 gamma.

21. The human cell or precursor thereof of any one of claims 10-20, wherein said human immune cells comprise NK cells, cytotoxic T cells and B cells.

22. The human cell or precursor thereof of any one of the above claims, further comprising a conditionally-expressed gene that causes apoptosis in the cell.

23. A method for treating an autoimmune disease in a subject, comprising administering to the subject a population of human genetically modified cells or precursors according to any one of claims 1-22.

24. The method of claim 23, wherein the human genetically modified cells or precursors comprise stem cells.

25. A composition for use in treating an autoimmune disease, the composition comprising a human genetically modified cell or precursor thereof according to any one of claims 1-22.

26. The composition of claim 25, wherein the human genetically modified cell or precursor is a stem cell.

27. The composition of claim 25 or 26, wherein the autoimmune disease is a localized autoimmune disease.

28. The composition of claim 27, where in the localized autoimmune disease is one of selected from Graves' Disease, Crohn's Disease, Addison's Disease, autoimmune hepatitis, Hashimoto's Thyroiditis, Reactive Arthritis, Giant-cell Arteritis (GCA), and celiac disease.

29. The composition of any one of claims 25-28, wherein the autoimmune disease is not type 1 diabetes or multiple sclerosis.
